(19) United States
(12) Patent Application Publication
(10) Pub. No.: US 2024/0043611 A1
(43) Pub. Date: Feb. 8, 2024

(54) POLYESTER POLYOLS COMPRISING ISOSORBIDE, ISOIDIDE OR ISOMANNIDE-DERIVED UNITS

(71) Applicant: BASF SE, Ludwigshafen am Rhein (DE)

(72) Inventors: Florian Ludwig GEYER, Ludwigshafen am Rhein (DE); Sedastian ROLLER, Ludwigshafen am Rhein (DE); Elena WOLF, Ludwigshafen am Rhein (DE); Steffen MADER, Ludwigshafen am Rhein (DE); Sylvester GROESSL, Ludwigshafen am Rhein (DE)

(21) Appl. No.: 18/265,991

(22) PCT Filed: Dec. 15, 2021
(86) PCT No.: PCT/EP2021/085843

§ 371 Date: Jun. 8, 2023

(30) Foreign Application Priority Data
Dec. 18, 2020 (EP) 20215282.3

(51) Int. Cl. C08G 63/685; C09D 175/06; C08G 18/24; C08G 18/79; C08G 18/42

(52) U.S. Cl. CPC C08G 63/6854; C09D 175/06; C08G 18/246; C08G 18/792; C08G 18/42

(57) ABSTRACT

The present invention relates to polyester polyols comprising units derived from
a) at least one component (A) carrying at least one COOH group or a derivative thereof, wherein component (A) comprises
   (i) at least one compound carrying two COOH groups or derivatives thereof (A1), and
b) at least one component (B) carrying at least one OH group and no COOH group, wherein component (B) comprises
   (ii) at least one compound or oligomer carrying at least three OH groups and no COOH group (B1),
   (iii) at least one compound carrying two OH groups (B2) selected from the group consisting of

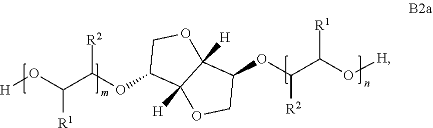

B2a and B2c,
wherein n and m are independently from each other 0 or 1, and R¹ and R² are independently selected from the group consisting of H, CH₃ and CH₂CH₃, and
(iv) optionally at least one compound, oligomer or polymer carrying two OH groups and no COOH group, which is different from B2 (B3),
wherein the molar ratio of the OH groups of components B1 to the sum of OH groups of components B1, B2 and B3 is in the range of from 25 to 90%,
and to an organic solvent-based two-component coating composition comprising
a) a first component (K1) comprising (i) at least one polyester polyol of the present invention, and (ii) optionally at least one polymer carrying more than one OH group, which is different from the polyester polyol of the present invention, (D), and
b) a second component (K2) comprising (i) at least one compound, oligomer or polymer carrying more than one N═C═O group or blocked N═C═O group (F),
to substrates coated with the coating compositions, and to coating layers on a substrate formed from the coating compositions.

The present invention relates to polyester polyols comprising isosorbide, isoidide or isomannide-derived units, to solutions comprising the polyester polyols, to organic solvent-based two component coating compositions suitable for yielding polyurethane coatings comprising the polyester polyols, and to substrates coated with the coating composition.

Organic solvent-based two-component coating compositions suitable for yielding polyurethane coatings are widely used in various applications, for example as coating composition for automotive and industrial coatings.

Monomers, which are derived from renewable resources, so-called “biomonomers”, represent a renewable alternative to the use of monomers derived from fossil resources in the preparation of polymers.

The use of isosorbide as biomonomer in organic solvent-based two-component coating compositions suitable for yielding a polyurethane coating is known in the art.

WO2019081867, WO2019081868 and WO2019081869 describe coating composition comprising isosorbide, at least one other polyol selected from the group consisting of polyester polyol, polyether polyol and polycarbonate polyol, and a polyisocyanate.

S. Opera, V.-O. Ptolinca, V. Opera, Eur. Polym. J. 2016, 83, 161-172 describes coating compositions comprising isosorbide, polytetramethylene ether glycol and hexamethylene diisocyanate.

B. A. J. Noordover, V. G. van Staalduinen, R. Duchateau, C. E. Koning, R.A.T.M. van Benthen, M. Mak, A. Heise, A.E. Frissen, J. van Harveren, Biomacromolecules 2006, 7, 3406-3416 describes polyester polyols obtained from monomers comprising succinic acid and isosorbide and optionally 2,3-butandiol, 1,3-propandiol or 1,1,1-trimethylolpropane, and also polyurethane coatings obtained from the polyester polyol and various polyisocyanates.

EP3660072A does not relate to coating compoitions, but to adhesive compositions comprising polyester polyols obtainable from a reaction mixture comprising isosorbide, at least one dicarboxylic acid and at least one triglyceride selected from or derived from ester of glycerol, as well as at least one NCO-terminated compound.

EP2325229A1 describes polyesters obtained from terephthalic or isophthalic acid, ethylene glycol, a dianhydrohexital, such as isosorbide, and one or more linar chain dicarboxylic acid.

US2014464530A1 describes polyesters carrying (meth)acryloyl groups obtained from polyols, polyacids and (meth)acrylating compounds.

Organic solvent-based two-component coating compositions suitable for yielding a polyurethane coating should ideally have a good drying behavior, and the coatings formed from the organic solvent-based two-component coating composition should show good mechanical properties.

It was the object of the present invention to provide organic solvent-based two-component compositions suitable for yielding polyurethane coatings at least partly derived from biomonomers, which compositions show a good drying behavior, in particular a short cotton wool drying time.

This object is solved by the polyester polyols of claim 1, the solution comprising the polyester-polyols of claim 12, the organic solvent-based two component coating composition of claim 13, the substrate of claim 16, and the coating layer on a substrate of claim 17.

The polyester polyols of the present invention are polyester polyols comprising units derived from

a) at least one component (A) carrying at least one COOH group or a derivative thereof, wherein component (A) comprises
(i) at least one compound carrying two COOH groups or a derivative thereof (A1), and

b) at least one component (B) carrying at least one OH group and no COOH group, wherein component (B) comprises
(ii) at least one compound or oligomer carrying at least three OH groups and no COOH group (B1),
(iii) at least one compound carrying two OH groups (B2) selected from the group consisting of

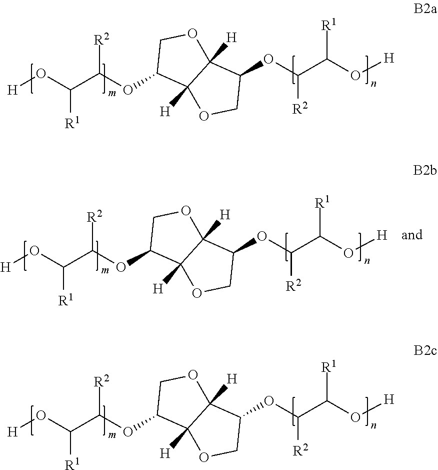

wherein n and m are independently from each other 0 or 1, and R¹ and R² are independently selected from the group consisting of H, CH₃ and CH₂CH₃, and
(iv) optionally at least one compound, oligomer or polymer carrying two OH groups and no COOH group, which is different from B2 (B3),
   wherein the molar ratio of the OH groups of components B1 to the sum of OH groups of components B1, B2 and B3 is in the range of 25 to 90%.

The polyester polyols of the present invention are preferably polyester polyols obtainable by reaction of

a) at least one component (A) carrying at least one COOH group or a derivative thereof, wherein component (A) comprises
(i) at least one compound carrying two COOH groups or a derivative thereof (A1), and

b) at least one component (B) carrying at least one OH group and no COOH group, wherein component (B) comprises
(ii) at least one compound or oligomer carrying at least three OH groups and no COOH group (B1),
(iii) at least one compound carrying two OH groups (B2) selected from the group consisting of

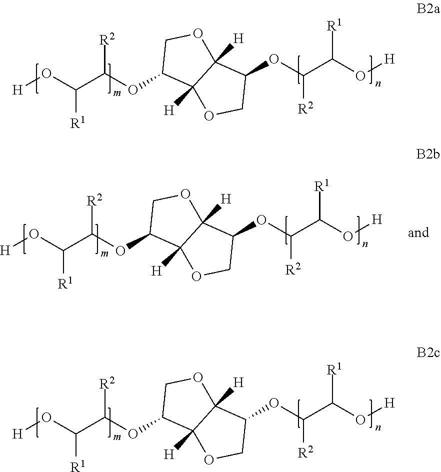

wherein n and m are independently from each other 0 or 1, and R₁ and R₂ are independently selected from the group consisting of H, CH₃ and CH₂CH₃, and
(iv) optionally at least one compound, oligomer or polymer carrying two OH groups and no COOH group, which is different from B2 (B3),

wherein the molar ratio of the OH groups of components B1 to the sum of OH groups of components B1, B2 and B3 is in the range of 25 to 90%.

The molar ratio of the OH groups of components B1 to the sum of OH groups of components B1, B2 and B3 is in the range of 25 to 90% means that the equivalent ratio of the OH groups of components B1 to the OH groups of components B1, B2 and B3 is in the range of 0.25/1 to 0.90/1.

The components (A) carrying at least one COOH group or a derivative thereof are preferably aliphatic or alicyclic components carrying at least one COOH group or a derivative thereof.

Preferably, none of the components (A) or (B) carries acryloyl or methacryloyl groups.

The compound carrying two COOH groups or a derivative thereof (A1) can also carry at least one group independently selected from the group consisting of OH group and NH₂ group.

Compounds carrying two COOH groups or a derivative thereof (A1) have preferably a molecular weight of below 500 g/mol, and most preferably of below 250 g/mol.

Compounds carrying two COOH groups or derivatives thereof (A1) can be an aliphatic, alicyclic or aromatic compound carrying two COOH groups or derivatives thereof. Preferred compounds carrying two COOH groups or derivatives thereof (A1) are aliphatic or alicyclic compounds carrying two COOH groups or derivatives thereof.

Aromatic compounds carrying two COOH groups are compounds carrying two COOH groups, wherein at least one COOH group is directly attached to an aromatic ring. Alicyclic compounds carrying two COOH groups are compounds carrying two COOH groups, which comprise at least one alicyclic ring and wherein each COOH group is not directly attached to an aromatic ring. Aliphatic compounds carrying two COOH groups are compounds carrying two COOH groups, which comprise no alicyclic ring, and wherein each COOH group is not directly attached to an aromatic ring. Preferred aliphatic and alicyclic compounds carrying two COOH groups or derivatives thereof do not carry aromatic rings.

Derivatives of the compounds carrying two COOH groups can be (i) the corresponding anhydride in monomeric or polymeric form, (ii) the corresponding mono- or di-C₁₋₄-alkyl esters such as monomethyl ester, dimethyl ester, monoethyl ester, diethyl ester or mixed methyl ethyl esters (iii) the corresponding amides, or (iv) the corresponding acid halides such as chlorides or bromides

Examples of C₁₋₄-alkyl are methyl, ethyl, propyl, isopropyl, n-butyl, sec-butyl and tert-butyl.

Preferred derivatives of component (A1) are (i) the corresponding anhydride in monomeric form or (ii) the corresponding mono- or di-C₁₋₄-alkyl esters.

Examples of aliphatic compounds carrying two COOH groups are oxalic acid, malonic acid, succinic acid, glutaric acid, adipic acid, pimelinic acid, suberic acid, azelaic acid, sebacic acid, 1,11-undecanedicarboxylic acid, 1,12-dodecanedicarboxlylic acid, maleic acid, fumaric acid, 2-methylmalonic acid, 2-ethylmalonic acid, 2-methylsuccinic acid, 2-ethylsuccinic acid, itaconic acid, 3,3-dimethylglutaric acid, 2-phenylmalonic acid 2-phenylsuccinic acid, glutamic acid, aspartic acid, tartaric acid and malic acid.

Examples of alicyclic compounds carrying two COOH groups are cyclopentane-1 ,2-dicarboxylic acid, cyclopentane-1,3-dicarboxylic acid, cyclohexane-1,2-dicarboxylic acid, cyclohexane-1,3-dicarboxylic acid, cyclohexane-1,4-dicarboxylic acid, cycloheptane-1,2-dicarboxylic acid, 1,2-bis(carboxymethyl)-cyclohexane, 1,3-bis(carboxymethyl)-cyclohexane and 1,4-bis(carboxymethyl)-cyclohexane.

Examples of aromatic compounds carrying two COOH groups are 2-5-furandicarboxylic acid, phthalic acid, isophthalic acid, terephthalic acid and bis(4-carboxyphenyl) methane.

Preferably, compound A1 is at least one aliphatic or alicyclic compound carrying two COOH groups or a derivative thereof. More preferably, compound A1 is at least one alicyclic compound carrying two COOH groups or derivatives thereof. Even more preferably, compound A1 is at least one alicyclic compound carrying two COOH groups independently selected from the group consisting of cyclohexane-1,2-dicarboxylic acid, cyclohexane-1,3-dicarboxylic acid, cyclohexane-1,4-dicarboxylic acid and derivatives thereof. Most preferably, compound A1 is cyclohexane-1,2-dicarboxylic acid or a derivative thereof. In particular, compound A1 is cyclohexane-1,2-dicarboxylic acid anhydride.

Component A can comprise further components carrying at least one COOH group or a derivative thereof, which are different from component A1, for example compounds carrying at least three COOH groups or derivatives thereof (A2) and compounds carrying only one COOH group or derivatives thereof (A3).

The further components A such as A2 and A3 can also optionally carry at least one group independently selected from the group consisting of OH group and NH₂ group.

Derivatives thereof are as defined above.

Examples of compounds carrying at least three COOH groups or derivatives thereof (A2) are 1,3,5-cyclohexanetricarboxylic acid, cis- and trans-aconitic acid, citric acid, isocitric acid, tricarballylic acid, 1,2,4-benzenetricarbocxylic acid, 1,3,5-benzenetricarbocxylic acid, 1,2,4,5-benzenetetracarboxylic acid, mellitic acid and pyromellitic dianhydride.

Examples of compounds carrying one COOH (A3) or derivatives thereof (A2) are dimethylolpropionic acid or dimethylolbutyric acid.

The compound or oligomer carrying at least three OH groups and no COOH group (B1) has preferably a molecular weight of below 1500 g/mol, more preferably 1000 g/mol, most preferably of below 500 g/mol.

The compound or oligomer carrying at least three OH groups and no COOH group (B1) can also carry heteroatom-containing groups such as isocyanurate, ester or ether groups.

Examples of compounds or oligomers carrying at least three OH groups and no COOH group (B1) are compounds or oligomers carrying three OH groups and no COOH group and compounds or oligomers carrying at least four OH groups and no COOH group.

Examples of compounds or oligomers carrying three OH groups and no COOH group are glycerol, butane-1,2,3-triol, butane-1,2,4-triol, pentane-1,2,3-triol, pentane-1,2,4-triol, pentane-1,2,5-triol, hexane-1,2,3-triol, hexane-1,2,4-triol, hexane-1,2,5-triol, hexane-1,2,6-triol, hexane-1,3,4-triol, hexane-1,3,5-triol, hexane-1,3,6-triol, hexane-1,4,5-triol, tetrahydrofuran-2,3,4-triol, tetrahydrofuran-2,3,5-triol, 2-(hydroxymethyl)tetrahydrofuran-3,4-diol, 5-(hydroxymethyl)tetrahydrofuran-2,4-diol, (3-hydroxytetrahydrofuran-2,5-diyl)dimethanol, 1-(3-hydroxytetrahydrofuran-2-yl)ethane-1,2-diol, 1-(4-hydroxytetrahydrofuran-2-yl)ethane-1,2-diol, 2-(2- hydroxyethyl)tetrahydrofuran-3,4-diol, 2-(1-hydroxyethyl)tetrahydrofuran-3,4-diol, 6-(hydroxymethyl)tetrahydro-2H-pyran-2,3-diol, 6-(hydroxymethyl)tetrahydro-2H-pyran-2,4-diol, 6-(hydroxymethyl)tetrahydro-2H-pyran-2,4-diol, 6-(hydroxymethyl)tetrahydro-2H-pyran-2,5-diol, 1,3,5-tris(hydrownethyhisocyanurate, 1,3,5-tris(2-hydroxyethyl)isocyanurate, 1,3,5-tris(2-hydroxyisopropyl)isocyanurate, 1,3,5-tris(2-hydroxypropyl)isocyanurate, 1,3,5-tris(2-hydroxybutyl)isocyanurate, trimethylolmethane, 1,1,1-trimethylolethane and 1,1,1-trimethylolpropane, as well as ethoxylated, propoxylated and/or butoxylated derivatives thereof.

Examples of compounds or oligomers carrying at least four OH groups and no COOH group are pentane-1,2,3,4-tetraol, pentane-1,2,3,5-tetraol, pentane-1,2,4,5-tetraol, hexane-1,2,3,4-tetraol, hexane-1,2,3,6-tetraol, hexane-1,2,5,6-tetraol, hexane-1,2,4,6-tetraol, hexane-1,2,3,5-tetraol, hexane-1,2,3,5-tetraol, tetrahydrofuran-2,3,4,5-tetraol, 5-(hydroxymethyl)tetrahydrofuran-2,3,4-triol, 2,5-bis(hydroxymethyl)tetrahydrofuran-3,4-diol, 2-(1,2-dihydroxyethyhtetrahydrofuran-3,4-diol, 6-(hydroxymethyl)tetrahydro-2H-pyran-2,3,5-triol, 6-(hydroxymethyl)tetrahydro-2H-pyran-2,3,4-triol, 6-(hydroxymethyl)tetrahydro-2H-pyran-2,4,5-triol, pentaerythritol, di(pentaerythritol), diglycerol, polyglycerol, di(trimethylolpropane), inositol, sugars such as glucose, fructose and sucrose, sugar alcohols such as sorbitol, mannitol, threitol, erythritol, adonitol (ribitol), arabitol (lyxitol), xylitol, dulcitol (galactitol), malitol and isomalt, as well as ethoxylated, propoxylated and/or butoxylated derivatives thereof.

Preferably, the compounds or oligomers carrying at least three OH groups and no COOH group (B1) are compounds or oligomers carrying three OH groups and no COOH group. More preferably, the compounds or oligomers carrying at least three OH groups and no COOH group (B1) are 1,3,5-tris(hydroxymethyl)isocyanurate, 1,3,5-tris(2-hydroxyethyl)isocyanurate, 1,3,5-tris(2-hydroxyisopropyl)isocyanurate, 1,3,5-tris(2-hydroxpropyl)isocyanurate ,1,3,5-tris(2-hydroxy-butyl)isocyanurate, trimethylolmethane, 1,1,1-trimethylolethane or 1,1,1-trimethylolpropane, as well as ethoxylated, propoxylated or butoxylated derivatives thereof. Even more preferably, the compounds or oligomers carrying at least three OH groups and no COOH group (B1) are 1,3,5-tris(2-hydroxyethyl)isocyanurate or 1,1,1-trimethylolpropane, as well as ethoxylated, propoxylated or butoxylated derivatives thereof. Most preferably, the compounds or oligomers carrying at least three OH groups and no COOH group (B1) are 1,3,5-tris(2-hydroxyethyl)isocyanurate.

Preferably, the compound carrying two OH groups (B2) is a compound of formula

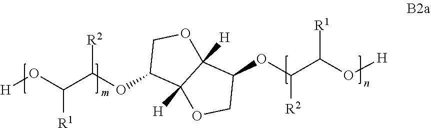

wherein n and m are independently from each other 0 or 1, and R¹ and R² are independently selected from the group consisting of H, CH₃ and CH₂CH_{3.}

More preferably, the compound carrying two OH groups (B2) is a compound of formula

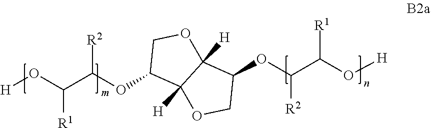

wherein n and m are independently from each other 0 or 1, and R¹ and R² are independently selected from the group consisting of H, CH³ and CH² CH³, with the proviso that at least one of R 1 or R² is H.

More preferably, the compound carrying two OH groups (B2) is a compound of formula

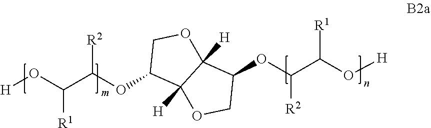

wherein n and m are independently from each other 0 or 1, and R¹ and R² are H. Most preferably, the compound carrying two OH groups (B2) is a compound of formula

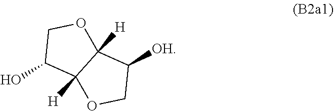

Compound B2a1 is also called isosorbide.

Isosorbide can be obtained by methods known in the art, for example by acid-catalyzed dehydration of D-sorbitol as described in DE488602. D-Sorbitol can be obtained by catalytic hydrogenation of D-glucose, which in turn can be produced by hydrolysis of starch as described in M. Rose, R. Palkovits, ChemSusChem. 2012, 5, 167-176.

The compound of formula B2b, wherein n and m are 0, is also called isoidide.

Isoidide can be obtained by methods known in the art, for example by isomerization of isosorbide followed by distillative isolation as described in examples 1 to 8 of WO2013125950, followed by distillative isolation as described in example 9 of WO2013125950.

The compound of formula B2c, wherein n and m are 0, is also called isomannide.

Isomannide can be prepared by methods known in the art, for example by dehydration of D-mannitol, which in turn can be obtained by catalytic hydrogenation of D-fructose. D-fructose can be produced by hydrolysis of starch. Isomannide can also be prepared by oxidation of isosorbide as described in the examples of WO2010089223 followed by reduction of the obtained diketo compound as described in example 1 to 5 of WO2018112774.

Isosorbide, isoidide and isomannide can be modified with ethylene oxide, propylene oxide or butylene oxide or with derivatives of ethylene glycol, propylene glycol or butylene glycol such as ethylene carbonate, propylene carbonate and butylene carbonate to yield the ethoxylated, propoxylated or butoxylated derivatives by methods known in the art for the ethoxylation, propoxylation and butoxylation of alcohols in order to yield the compounds of formula B2a, B2b and B2c, wherein at least n or m are 1.

The preparation of the compound of formula 2Ba, wherein n and m are 1 and R¹ and R² are H, from isosorbide and ethylene carbonate is described in example 1 of U.S. Pat. No. 6,608,167B1.

The preparation of the compound of formula 2Bc, wherein n and m are 1 and R¹ and R² are H, from isomannide and an ethyleneglycol derivative is exemplified in P.R. Ashton, A.M. Heiss, D. Pasini, F. M. Raymo, A. N. Shipway, J. F. Stoddart, N. Spencer, Eur. J. Org. Chem. 1999, 5, 995-1004.

The compound, oligomer or polymer carrying two OH groups and no COOH group, which is different from B2, (B3) preferably has a molecular weight of below 1000 g/mol, more preferably of below 500 g/mol, and most preferably of below 250 g/mol.

The compound, oligomer or polymer carrying two OH groups and no COOH group, which is different from B2, (B3) can also carry isocyanurate, ester or ether groups.

The compound, oligomer or polymer carrying two OH groups and no COOH group, which is different from B2, (B3) is preferably an aliphatic or alicyclic compound carrying two OH groups and no COOH group, a polyether diol or a polyester diol.

Alicyclic compounds carrying two OH groups and no COOH group are compounds carrying two OH groups, which comprise at least one alicyclic ring and wherein each OH group is not directly attached to an aromatic ring. Aliphatic compounds carrying two OH groups and no COOH group are compounds carrying two OH groups, which comprise no alicyclic ring, and wherein each OH group is not directly attached to an aromatic ring. Preferred aliphatic and alicyclic compounds carrying two OH groups and no COOH group, do not comprise aromatic rings.

Examples of aliphatic compounds carrying two OH groups and no COOH group, which are different from B2, are ethylene glycol, propane-1,2-diol, propane-1,3-diol, butane-1,2-diol, butane-1,3-diol, butane-1,4-diol, butane-2,3-diol, pentane-1,2-diol, pentane-1,3-diol, pentane-1,4-diol, pentane-1,5-diol, pentane-2,3-diol, pentane-2,4-diol, hexane-1,2-diol, hexane-1,3-diol, hexane-1,4-diol, hexane-1,5-diol, hexane-1,6-diol, hexane-2,5-diol, heptane-1,2-diol, heptane-1,7-diol, octane-1,8-diol, octane-1,2-diol, nonane-1,9-diol, decane-1,2-diol, decane-1,10-diol, dodecane-1,2-diol, dodecane-1,12-diol, hexa-1,5-diene-3,4-diol, neopentyl glycol, 2-methyl-pentane-2,4-diol, 2,4-dimethyl-pentane-2,4-diol, 2-ethyl-hexane-1,3-diol, 2,5-dimethyl-hexane-2,5-diol, 2,2,4-trimethyl-pentane-1,3-diol, pinacol, 2,5-bis(hydroxymethyl)tetrahydrofuran, tetrahydrofuran-2,3-diol, tetrahydrofuran-2,4-diol, tetrahydrofuran-2,5-diol, 5-(hydroxymethyl)tetrahydrofuran-2-ol, 5-(hydroxymethyl)tetrahydrofuran-3-ol, 2-(hydroxymethyl)tetrahydrofuran-3-ol, 2-ethyltetrahydrofuran-3,4-diol, 2-(2-hydroxyethyl)tetrahydrofuran-3-ol, 5-(2-hydroxyethyl)tetrahydrofuran-3-ol, 2-(1-hydroxyethyl)tetrahydrofuran-3-ol, 5-(1-hydroxyethyl)tetrahydrofuran-3-ol, 6-(hydroxymethyhtetrahydro-2H-pyran-2-ol, 6-(hydroxymethyl)tetrahydro-2H-pyran-3-ol, 2-(hydroxymethyl)tetrahydro-2H-pyran-4-ol, 2-(hydroxymethyl)tetrahydro-2H-pyran-3-ol and hydroxypivalinic acid neopentyl glycol ester.

Examples of alicyclic compounds carrying two OH groups and no COOH group, which are different from B2, are 2,2,4,4-tetramethyl-1,3-cyclobutandiol, cyclopentane-1,2-diol, cyclopentane-1,3-diol, 1,2-bis(hydroxymethyl) cyclopentane, 1,3-bis(hydroxymethyl) cyclopentane, cyclohexane-1,2-diol, cyclohexane-1,3-diol, cyclohexane-1,4-diol, 1,1-bis(hydroxymethyl)-cyclohexane, 1,2-bis(hydroxymethyl)-cyclohexane, 1,3-bis(hydroxymethyl)-cyclohexane, 1,4-bis(hydroxymethyl)-cyclohexane, 1,1-bis(hydroxyethyl)-cyclohexane, 1,2-bis(hydroxyethyl)cyclohexane, 1,3-bis(hydroxyethyl)cyclohexane and 1,4-bis(hydroxyethyl)cyclohexane, cycloheptane-1,3-diol, cycloheptane-1,4-diol and cycloheptane-1,2-diol.

Examples of polyether diols are diethylene glycol, triethylene glycol, dipropylene glycol, tripropylene glycol, polyethylene glycols HO(CH₂CH₂O)ₙ—H, polypropylene glycols HO(CH(CH₃)—CH₂—O)ₙ—H, n being an integer and n>=4, polyethylene-polypropylene glycols, the sequence of the ethylene oxide or propylene oxide units being blockwise or random, polytetramethylene glycols, and polytetrahydrofuran.

An example of a polyester diol is polycaprolactone prepared from caprolactone and a diol.

The component (B) can comprise further components carrying at least one OH group and no COOH group, which are different from B1, B2 and B3, for example compounds carrying only one OH group and no COOH group (B4).

Examples of compounds carrying only one OH group and no COOH group (B4) are methanol, ethanol, 1-propanol, isopropanol, 1-butanol, sec-butanol, isobutanol, tert-butanol, 1-pentanol, 3-methylbutan-2-ol, 2-methylbutan-2-ol, fatty alcohols such as 1-hexanol, 1-heptanol, 1-octanol, 1-nonanol, 1-decanol, 1-undecanol, 1-dodecanol, 1 tetradecanol, 1-hexadecanol, 1-octade-canol, cis-9-hexadecen-1-ol, cis-9-octadecen-1-ol, cis,cis-9,12-octadecadien-1-ol, 6,9,12-octa-decatrien-1-ol, 1-methoxpropan-2-ol, cyclopentanol, cyclohexanol, cycloheptanol, 1-methylcy-clopentan-1-ol, 1-methylcyclohexan-1-ol, poly(ethylene gycol) monomethyl ether, poly(propyl-ene gycol) monomethyl ether, furfuryl alcohol and tetrahydrofurfuryl alcohol.

Preferably, less than 5 weight % of the polyester polyols of the present invention consists of units derived from compounds B4.

Preferably, at least 60 weight % of the polyester polyols of the present invention consists of units derived from compounds A1, B1 and B2. More preferably, at least 75 weight % of the polyester polyols of the present invention consists of units derived from compounds A1, B1 and B2. Even more preferably, at least 90 weight % of the polyester polyols of the present invention consists of units derived from compounds A1, B1 and B2. Most preferably, at least 95 weight % of the polyester polyols of the present invention consists of units derived from compounds A1, B1 and B2.

In particular, the polyester polyol of the present invention consists of units derived from compounds A1, B1 and B2.

The molar ratio of the OH groups of components B1 to the sum of OH groups of components B1, B2 and B3 is preferably in the range of 40% to 90% more preferably in the range of 50% to 90%, even more preferably in the range of 60 to 85% and most preferably in the range of 60 to 80%, meaning that the equivalent ratio of the OH groups of components B1 to the OH groups of components B1, B2 and B3 is preferably in the range of 0.4/1 to 0.9/1 more preferably in the range of 0.5/1 to 0.9/1, even more preferably in the range of 0.6/1 to 0.85/1 and most preferably in the range of 0.6/1 to 0.8/1.

The molar ratio of the sum of OH groups of all components A and B to the sum of COOH groups of all components A is preferably in the range of 1.05/1 to 5/1, more preferably in the range of 1.05/1 to 3/1, even more preferably in the range of 1.05/1 to 2/1, and most preferably in the range of 1.10/1 to 1.7/1, meaning that the equivalent ratio of the OH groups of all components A and B to the COOH groups of all components A is preferably in the range of 1.05/1 to more preferably in the range of 1.05/1 to 3/1, even more preferably in the range of 1.05/1 to 2/1, and most preferably in the range of 1.10/1 to 1.7/1.

Preferably, the components A do not carry OH groups. In this case, the equivalent ratio of the OH groups of all components B to the COOH groups of all components A is preferably in the range of 1.05/1 to 5/1, more preferably in the range of 1.05/1 to 3/1, even more preferably in the range of 1.05/1 to 2/1, and most preferably in the range of 1.10/1 to 1.7/1.

The molar ratio of the OH groups of B2 to the sum of OH groups of B2 and B3, if present, is preferably in the range of 50% to 100%, more preferably in the range of 70% to 100%, even more preferably in the range of 90% to 100%, most preferably in the range of 95% to 100%, meaning that the equivalent ratio of the OH groups of B2 to the OH groups of B2 and B3, if present, is preferably in the range of 0.5/1 to 1/1 more preferably in the range of 0.7/1 to 1/1 even more preferably in the range of 0.9/1 to 1/1 most preferably in the range of 0.95/1 to 1/1.

The polyester polyols of the present invention preferably not comprise units derived from aromatic components carrying at least one COOH group or a derivative thereof.

The polyester polyols of the present invention preferably not comprise units deriving from glycerol or from derivatives thereof. Examples of derivatives of glycerol are esters of glycerol such as triesters of glycerol.

Preferably, the polyester polyols of the present invention do not carry acryloyl or methacryloyl groups.

The polyester polyols of the present invention are preferably so-called “hyperbranched” polyester polyols. “Hyperbranched” polyester polyols are defined to be polyester polyols of tree-like structure comprising non-terminal monomer units derived from components, which have at least three groups individually selected from the group consisting of OH group and COOH group or a derivative thereof (such as B1), wherein at least one of these groups has not reacted to form a linkage between two monomer units. Preferably, the molar ratio of non-terminal monomer units derived from components, which have at least three groups individually selected from the goup consisting of OH group and COOH group or derivative thereof (such as B1), wherein at least one of these groups has not reacted to form a linkage between two monomer units to non-terminal monomer units derived from components, which have at least three groups individually selected from the goup consisting of OH group and COOH group or derivative thereof (such as B1), wherein all of these groups have reacted to form a linkage between two monomer units is at least 5/95, more preferably at least 10/90, even more preferably at least 30/70. This molar ratio can be determined by methods known in the art, for example ¹³C-NMR or titration.

The polyester polyols of the present invention preferably have a hydroxyl number in the range of 50 to 400 mg KOH/g, more preferably in the range of 60 to 300 mgKOH/g, most preferably in the range of 70 to 230 mg KOH/g. The hydroxyl number is determined according to DIN 53240, 2016.

The polyester polyols of the present invention preferably have an acid number in the range of 1 to 200 mg KOH/g, more preferably in the range of 10 to 150 mg KOH/g, and most preferably in the range of 20 to 130 mg KOH/g. The acid number is determined according to DIN 53402, 1990.

The polyester polyols of the present invention preferably have a number average molecular weight Mn in the range of 400 to 10000 g/mol, more preferably in the range of 400 to 5000 g/mol, even more preferably in the range of 400 to 2000 g/mol and most preferably in the range of 400 to 1800 g/mol. The number average molecular weight Mn is determined using gel permeation chromatography calibrated to a polystyrene standard.

The polyester polyols of the present invention preferably have a weight average molecular weight Mw in the range of 400 to 30000 g/mol, more preferably in the range of 400 to 15000 g/mol, even more preferably in the range of 500 to 8000 g/mol and most preferably in the range of 600 to 8000 g/mol. The weight average molecular weight Mn is determined using gel permeation chromatography calibrated to a polystyrene standard.

The polyester polyols of the present invention preferably have a polydispersity Mw/Mn in the range of 1.1/1.0 to 20.0/1.0, more preferably in the range of 1.2/1.0 to 10.0/1.0 and most preferably in the range of 1.3/1.0 to 5/1.0.

The polyester polyol can have a glass transition temperature (Tg) in the range of 10 to 100° C., preferably in the range of 20 to 50° C.

Also part of the invention is a process for the preparation of the polyester polyols of the present invention, which process comprises the step of reacting

a) at least one component (A) carrying at least one COOH group or a derivative thereof, wherein component (A) comprises
(i) at least one compound carrying two COOH groups or a derivative thereof (A1), with

b) at least one component (B) carrying at least one OH group and no COOH group, wherein component (B) comprises
(ii) at least one compound or oligomer carrying at least three OH groups and no COOH group (B1),
(iii) at least one compound carrying two OH groups (B2) selected from the group consisting of

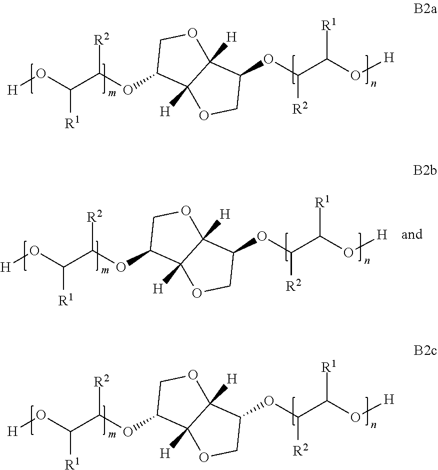

wherein n and m are independently from each other 0 or 1, and R¹ and R² are independently selected from the group consisting of H, CH₃ and CH₂CH₃, and
(iv) optionally at least one compound, oligomer or polymer carrying two OH groups and no COOH group, which is different from B2 (B3),
wherein the molar ratio of OH groups of components B1 to the sum of OH groups of components B1, B2 and B3 is in the range of 25 to 90%.

The molar ratio of the OH groups of components B1 to the sum of OH groups of components B1, B2 and B3 is in the range of 25 to 90% means that the equivalent ratio of the OH groups of components B1 to the OH groups of components B1, B2 and B3 is in the range of 0.25/1 to 0.90/1.

The reaction of components A1, B1, B2 and optionally B3 can be carried out in the presence or absence of solvent. Examples of suitable solvents include hydrocarbons such as n-heptane, cyclohexene, toluene, ortho-xylene, meta-xylene, para-xylene, xylene isomer mixture, ethylbenzene, chlorobenzene, ortho- and meta-dichlorobenzene. Of further suitability as solvents in the absence of acidic catalysts are ethers such as dioxane or tetrahydrofuran, and ketones such as methyl ethyl ketone and methyl isobutyl ketone. Preferably, the reaction is carried out in the absence of solvent.

Preferably, the water formed over the course of the reaction is removed continuously during the reaction. Water can be removed by distillation. Water can also be removed by stripping, which comprises passing a gas, which is inert under the reaction conditions, such as nitrogen, through the reaction mixture. Water can also be removed by performing the reaction in the presence of a water-removing agent such as MgSO₄ and Na₂SO₄. It is also possible to combine the described methods for removal of water. Preferably, water is removed by distillation, optionally in combination with other water-removal methods.

If other volatile components, for example methanol or ethanol, are also formed over the course of the reaction, these can also be removed by distillation or stripping.

Preferably the reaction is performed in the presence of a catalyst. The catalyst can be selected from the group consisting of acidic inorganic, acidic organic catalysts and organometallic catalysts or mixtures thereof. More preferably, the catalyst is an acidic organometallic catalyst, most preferably titanium(IV) tetra(n-butoxide).

Examples of acidic inorganic catalysts are sulfuric acid, sulfates and hydrogen sulfates such as sodium hydrogen sulfate, phosphoric acid, phosphonic acid, hypophosphoric acid, aluminium sulfate hydrate, alum, acidic silica gel (pH<=6, especially pH<=5) and acidic aluminium oxide.

Examples of acidic organic catalysts are organic compounds containing phosphate groups, sulfonic acid groups, sulfate groups or phosphonic acid groups, such as para-toluene sulfonic acid. Further examples of acidic organic catalysts are acidic ion exchangers such as polystyrene resins being crosslinked with divinylbenzene and containing sulfonic acid groups.

Examples of organometallic catalysts are organic aluminium catalysts such as tris(n-butyloxy)aluminium, tris(isopropyloxy)aluminium and tris(2-ethylhexoxy)aluminium, as well as organic titanium catalysts such as titanium(IV) tetra(n-butoxide), titanium(IV) tetra(isopropoxide) and titanium(IV) tetra(2-ethylhexoxide), organic tin catalysts such as dibutyltin oxide, diphenyltin oxide, dibutyltin dichloride, tin(I)di(n-octanoate), tin(II) di(2-ethylhexanoate), tin(II) laurate, dibutyltin diacetate, dibutyltin dilaurate, dibutyltin dimaleate and dioctyltin diacetate as well as organic zinc catalysts such as zinc acetate.

Preferably, the reaction is carried out under a gas, which is inert under the reaction conditions. Suitable inert gases include nitrogen, noble gases such as argon, carbon dioxide or combustion gases.

The reaction can be performed at a pressure in the range of 10 mbar to 10000 mbar, preferably at a pressure in the range of 10 to 2000 mbar, more preferably at a pressure in the range of 10 to 1200 mbar, most preferably at a pressure in the range of 300 to 1100 mbar.

The temperature is usually in the range of 60 to 250° C., preferably, in the range of 100 to 220° C. and more preferably in the range of 120 to 200° C. It is preferred that the temperature increases during the polyesterifcation reaction.

The reaction can be monitored by the titration of the acid number or hydroxyl number. Usually, the reaction is stopped, when the target acid number or hydroxyl number of the polyester polyol is reached, by cooling the reaction mixture, preferably to below 100° C., more preferably to below 90° C., and diluting the reaction mixture with an organic solvent, for example butyl acetate, to yield a solution of the polyester polyol in the organic solvent.

Usually, the solution of the polyester polyol in the organic solvent is used for the preparation of the organic solvent-based two-component coating composition.

However, the polyester polyol of the present invention can also be isolated, if desired, from the solution of the polyester polyol in the organic solvent, for example, by the addition of water and filtering off the precipitated polyester polyol.

Also part of the present invention are solutions comprising at least one polyester polyol of the present invention and at least one organic solvent. Suitable organic solvents are esters, ketones, amides, ethers and aromatic hydrocarbons and mixtures thereof.

Examples of esters of are ethyl acetate, butyl acetate, 1-methoxy-2-propyl acetate, 2-butoxy ethyl acetate (butyl gycol acetate), propylene glycol diacetate, ethyl 3-ethoxy propionate, 3-methoxybutyl acetate, butyldiglycol acetate and propylene carbonate. Examples of ketones are acetone, methyl ethyl ketone and methyl isobutyl ketone. Examples of amides are dimethylformamide (DMF) and N-methyl pyrrolidone (NMP). Example of ethers are glycol ethers such as dipropylene glycol dimethylether, and cyclic ethers such as tetrahydrofuran and 1,4-dioxane. Examples of aromatic hydrocarbons are xylene and solvent naphtha.

A preferred organic solvent is an ester or mixtures thereof. A more preferred organic solvent is an ester of a C₁₋₆-alkanoic acids with a C₁₋₆ -alkanol such as butyl acetate and ethyl acetate. The most preferred organic solvent is butyl acetate.

The solid content of the solution is preferably in the range of 30 to 90% by weight, more preferably 50 to 80% by weight, and most preferably in the range of 60 to 75% by weight.

The amount of polyester polyol of the present invention is preferably in the range of 30 to 90% by weight based on the weight of the solution, more preferably 50 to 80% by weight based on the weight of the solution, and most preferably in the range of 60 to 75% by weight based on the weight of the solution.

The viscosity of the solution is preferably in the range of 500 to 15000 mPa×s, more preferably, in the range of 1000 to 10000 mPa×s. The viscosity is determined using a cone plate viscosimeter set to a shear rate of 100 s⁻¹ at 23° C.

Also part of the present invention is an organic solvent-based two-component coating composition comprising
a) a first component (K1) comprising (i) the polyester polyol of the present invention, and (ii) optionally at least one polymer carrying more than one OH group, which is different from the polyester polyol of the present invention, (D)
   and
b) a second component (K2) comprising (i) at least one compound, oligomer or polymer carrying more than one N═C═O group or blocked N═C═O group (F).

Preferably, the polymer carrying more than one OH group, which is different from the polyester polyol of the present invention, (D) is present in the first component (K1).

The polymer carrying more than one OH group, which is different from the polyester polyol of the present invention, (D) has preferably a hydroxyl number in the range of 40 to 400 mg 40 KOH/g, more preferably in the range of 50 to 250 mgKOH/g, even more preferably in the range of 85 to 200 mg KOH/g. The hydroxyl number is determined according to DIN53240, 2016.

The polymer carrying more than one OH group, which is different from the polyester polyol of the present invention, (D) has preferably an acid number of less than 100 mg KOH/g, more preferably of less than 50 mgKOH/g, even more preferably of less than 20 mg KOH/g and most preferably of less than 15 mg KOH/g. The acid number is determined according to DIN53402, 1990.

The polymer carrying more than one OH group, which is different from the polyester polyol of the present invention, (D) can be selected from the group consisting of (meth)acrylic polymer carrying more than one OH group, polyester carrying more than one OH group, polyether carrying more than one OH group, urea-formaldehyde resin carrying more than one OH group, melamine-formaldehyde resins carrying more than one OH group, polycarbonate carrying more than one OH group polyurethane carrying more than one OH group, and polymers of ethylenically unsaturated monomers, excluding (meth)acrylic-type monomers, carrying more than one OH group.

(Meth)acrylic means either methacrylic and/or acrylic.

The (meth)acrylic polymer carrying more than one OH group can comprise monomer units derived from at least one (meth)acrylic monomer carrying at least one OH group, from at least one (meth)acrylic monomer carrying no OH groups, and optionally from other ethylenically unsaturated monomers.

Examples of (meth)acrylic monomers carrying at least one OH group are monoesters of (meth)acrylic acid with aliphatic diols, preferably C₁₋₁₀-aliphatic diols, more preferably C₁₋₄-aliphatic diols, such as 2-hydroxyethyl methacrylate, 2-hydroxyethyl acrylate, 2-hydroxypropyl methacrylate, 2-hydroxypropyl acrylate, 3-hydroxypropyl methacrylate, 3-hydroxypropyl acrylate, 4-hydroxybutyl methacrylate, 4-hydroxylbutyl acrylate, 6-hydroxyhexyl methacrylate and 6-hydroxyhexyl acrylate.

Examples of (meth)acrylic monomers carrying no OH group are C₁₋₂₀-alkyl (meth)acrylates such as methyl methacrylate, methyl acrylate, ethyl methacrylate, ethyl acrylate, butyl methacrylate, n-butyl acrylate, n-hexyl methacrylate, n-hexyl acrylate, n-heptyl methacrylate, n-heptyl acrylate, n-octyl methacylate, n-octyl acrylate, 2-ethyl hexyl methacrylate and 2-ethylhexyl acrylate, as well as C₅₋₇-cycloalkyl (meth)acrylates such as cyclohexyl methacrylate and cyclohexylacrylate, as well as other (meth)acrylate esters carrying no OH group such as isobornyl methacrylate and isobornyl acrylate.

Examples of C₁₋₂₀ -alkyl are methyl, ethyl, n-propyl, isopropyl, n-butyl, sec-butyl, tert-butyl, n-pentyl, iso-pentyl, sec-pentyl, neopentyl, 1,2-dimethylpropyl, isoamyl, n-hexyl, isohexyl, n-heptyl, isoheptyl, n-octyl, 2-ethylhexyl, trimethylpentyl, n-nonyl, n-decyl, n-undecyl and n-dodecyl.

Examples of C₅₋₇-cycloalkyl are cyclopentyl, cyclohexyl and cycloheptyl.

Further examples of (meth)acrylic monomers carrying no OH group are methacrylonitrile, acrylonitrile, methacrylic acid, acrylic acid, methacrylamide, acrylamide, N-(methoxymethyl)methacrylamide, N-(methoxymethyl)acrylamide, N-(2-methoxyethyl)methacrylamide, N-(2-methoxyethyl)acrylamide, N-(2-methoxypropyl)methacrylamide and N-(2-methoxypropyl)acrylamide.

Examples of other ethylenic unsaturated monomers are unsaturated C₂₋₈-aliphatic compounds such as ethylene, propylene, isobutylene, butadiene and isoprene, C₆₋₂₀-aromatic compounds carrying one vinyl group such as styrene, vinyl toluene, 2-n-butyl styrene, 4-n-butyl styrene and 4-n-decyl styrene, vinyl esters of saturated C₁₋₂₀-fatty acids such as vinyl acetate, vinyl propionate, vinyl stearate and vinyl laurate, alpha, beta -unsaturated carboxylic acids different from methacrylic acid and acrylic acid such as crotonic acid and their C₁₋₂₀-alkyl esters, nitriles and amides, ethylenic unsaturated diacids such as fumaric acid, itaconic acid and maleic acid as well as their anhydrides such as maleic anhydride, vinyl ethers of C₁₋₁₀-alcohols such as vinyl methyl ether, vinyl isobutyl ether, vinyl hexyl ether and vinyl octyl ether, vinyl amides such as N-vinyl formamide, N-vinyl pyrrolidone and N-vinyl caprolactam, as well as heteroaromatic compounds carrying one vinyl group such as N-vinyl imidazole.

Preferably, the polymer carrying more than one OH group, which is different from the polyester polyol of the present invention, (D) is at least one (meth)acrylic polymer carrying more than one OH group.

More preferably, the polymer carrying more than one OH group, which is different from the polyester polyol of the present invention, (D) is a (meth)acrylic resin polymer carrying more than one OH group and comprising monomer units derived from at least one (meth)acrylic monomer carrying at least one OH group selected from the group consisting of 2-hydroxyethyl methacrylate, 2-hydroxyethyl acrylate, 2-hydroxypropyl methacrylate, 2-hydroxypropyl acrylate, 3-hydroxypropyl methacrylate, 3-hydroxypropyl acrylate, 4-hydroxybutyl methacrylate and 4-hydroxylbutyl acrylate.

Most preferably, the polymer carrying more than one OH group, which is different from the polyester polyol of the present invention, (D) is a (meth)acrylic resin polymer carrying more than one OH group and comprising monomer units derived from at least one (meth)acrylic monomer carrying at least one OH group selected from the group consisting of 2-hydroxyethyl methacrylate and 2-hydroxyethyl acrylate.

The (meth)acrylic polymer carrying more than one OH group has preferably a number average molecular weight Mn in the range of 500 to 30000 g/mol, more preferably in the range of 500 to 10000g/mol, even more preferably in the range of 500 to 5000 g/mol. The number average molecular weight is determined using gel permeation chromatography calibrated to a polystyrene standard.

The (meth)acrylic polymer carrying more than one OH group has preferably a weight average molecular weight Mw in the range of 500 to 50000 g/mol, more preferably in the range of 500 to 10000 g/mol. The weight average molecular weight is determined using gel permeation chromatography calibrated to a polystyrene standard.

The polymer carrying more than one OH group, which is different from the polyester polyol of the present invention, (D) can be prepared by methods known in the art.

For example, (meth)acrylic polymers carrying more than one OH group comprising monomer units derived from at least one (meth)acrylic monomer carrying at least one OH group, from at least one (meth)acrylic monomer carrying no OH groups, and optionally from other ethylenic unsaturated monomers, can be prepared by radical polymerization of the corresponding monomers. The radical polymerization is usually performed in the presence of at least one radical initiator such as azobis(isobutyronitrile), dibenzoyl peroxide or sodium peroxodisulfate. The radical polymerization can be performed, in organic solution, or in bulk polymerization. The radical polymerization can be performed in a batch process or as continuous process.

Component (F) is at least one compound, oligomer or polymer carrying more than one N═C═O group or blocked N═C═O group.

Blocked N═C═O group are groups that can be de-blocked to release the N═C═O group under specific conditons, for example at elevated temperatures, such as at temperatures above 110° C. Compounds, oligomers or polymers carrying more than one blocked N═C═O groups can be prepared, for example, by reacting the corresponding compounds, oligomers or polymers carrying more than one N═C═O group with a compound carrying acidic hydrogens. Examples of compounds carrying acidic hydrogens are diethyl malonate, 3,5-dimethylpyrazole and 2-butanonoxime.

The compound carrying more than one N═C═O group or blocked N═C═O group is preferably an aliphatic, alicyclic or aromatic compound carrying at least two N═C═O groups or blocked N═C═O groups, for example an aliphatic, alicyclic or aromatic compound carrying two N═C═O groups or blocked N═C═O groups, or an aliphatic, alicyclic or aromatic compound carrying three N═C═O groups or blocked N═C═O groups.

Aromatic compounds carrying at least two N═C═O groups or blocked N═C═O groups are compounds carrying at least two N═C═O groups or blocked N═C═O groups, wherein at least one N═C═O group is directly attached to an aromatic ring. Alicyclic compounds carrying at least two N═C═O groups or blocked N═C═O groups are compounds carrying at least two N═C═O groups or blocked N═C═O groups, which comprise at least one alicyclic ring and wherein each N═C═O group is not directly attached to an aromatic ring. Aliphatic compound carrying at least two N═C═O groups or blocked N═C═O groups are compounds carrying at least two N═C═O groups or blocked N═C═O groups, which comprise no alicyclic ring, and wherein each N═C═O group is not directly attached to an aromatic ring. Preferred aliphatic, alicyclic and aromatic compounds carrying at least two N═C═O group or blocked N═C═O group, exclusively consist, apart from the N═C═O groups or blocked N═C═O groups, of carbons and hydrogens.

Examples of aliphatic compounds carrying two N═C═O groups are tetramethylene 1,4-diisocyanate, pentamethylene 1,5-diisocyanate, hexamethylene 1,6-diisocyanate, octamethylene 1,8-diisocyanate, decamethylene 1,10-diisocyanate, dodecamethylene 1,12-diisocyanate, tetradecamethylene 1,14-diisocyanate, methyl 2,6-diisocyanatohexanoate, ethyl 2,6-diisocyanatohexanoate, trimethylhexane diisocyanate or tetramethylhexane diisocyanate,

Examples of alicyclic compounds carrying two N═C═O groups are 1,4-diisocyanatocyclohexane, 1,3-diisocyanatocyclohexane, 1,2-diisocyanatocyclohexane, 4,4′-di(isocyanatocyclohexyl)methane, 2,4′-di(isocyanatocyclohexyl)methane, 1-isocyanato-3,3,5-trimethyl-5-(isocyanatomethyl)cyclohexane (isophorone diisocyanate), 1,3- bis(isocyanatomethyl)cyclohexane, 1,4-bis(isocyanatomethyl)cyclohexane, 2,4- diisocyanato-1-methylcyclohexane, 2,6-diisocyanato-1-methylcyclohexane, and 3(or 4),8(or 9)-bis (isocyanatomethyhtricyclo[5.2.1.0(2,6)]decane.

Examples of aromatic compounds carrying two N═C═O groups are 2,4-tolylene diisocyanate, 2,6-tolylene diisocyanate, m-xylylene diisocyanate, p-xylylene diisocyanate, 2,4′-diisocyanatodiphenylmethane, 4,4′-diisocyanatodiphenylmethane, 1,3-phenylene diisocyanate,1,4-phenylene diisocyanate, 1-chloro-2,4-phenylene diisocyanate, 1,5-naphthylene diisocyanate, diphenylene 4,4′-diisocyanate, 4,4′-diisocyanato-3,3′-dimethylbiphenyl, 3-methyldiphenylmethane 4,4′-diisocyanate, tetramethylxylylene diisocyanate, 1,4-diisocyanatobenzene and diphenyl ether 4,4′-diisocyanate.

Examples of aliphatic compounds carrying three N═C═O groups are 1,4,8-triisocyanatononane, 2′-isocyanatoethyl 2,6-diisocyanatohexanoate.

Examples of aromatic compounds carrying three N═C═O groups are 2,4,6-triisocyanatotoluene, triphenylmethane triisocyanate and 2,4,4′-triisocyanatodiphenyl ether.

Compounds carrying more than one N═C═O group can be prepared by methods known in the art, for example by treating the corresponding amines with phosgene.

Examples of oligomer or polymer carrying more than one N═C═O group or blocked N═C═O group are oligomers or polymers carrying more than one N═C═O group or blocked N═C═O group, which comprise at least one unit independently derived from the group consisting of aliphatic, alicylic and aromatic compounds carrying at least two N═C═O group. Aliphatic, alicylic or aromatic compounds carrying at least two N═C═O groups are as defined above.

Examples of oligomers or polymers carrying more than one N═C═O groups are also so-called “polymeric diphenyl diisocyanate”.

The N═C═O content of the oligomers or polymers carrying more than one N═C═O group or blocked N═C═O group can be in the range of 1 to 60%, more preferably in the range of 5 to 40%, even more preferably in the range of 15 to 30%, most preferably in the range of 20 to 25%.

The N═C═O content is the weight ratio of the N═C═O groups of the oligomer or polymer carrying more than one N═C═O group to the oligomer or polymer carrying more than one N═C═O group.

When determing the N═C═O content, the oligomer or polymer carrying more than one N═C═O group must be in de-blocked form. The N═C═O content can, for example, be determined by the following method: 10 mL of a 1 N solution of n-dibutyl amine in xylene is added to 1 g of a compound, oligomer or polymer dissolved in 100 mL of N-methylpyrrolidone. The resulting mixture is stirred at room temperature for five minutes. Then, the resulting reaction mixture is subjected to back titration using 1 N hydrochloric acid to measure the volume of the hydrochloric acid needed for neutralizing the unreacted n-dibutyl amine. This then reveals how much mol n-dibutyl amine reacted with N═C═O groups. The content of N═C═O is the weight of all N═C═O groups in 1 g of oligomer or polymer carrying more then one N═C═O group/1 g of oligomer or polymer carrying more than one N═C═O group. The weight of all N═C═O groups is “mol reacted n-dibutyl amine” multiplied by the molecular weight of N═C═O, which is 42 g/mol.

Preferably, component F is an oligomer or polymers carrying more than one N═C═O group or blocked N═C═O group.

More preferably, component F is at least one oligomer or polymer carrying more than one N═C═O group or blocked N═C═O group and comprising (i) at least one unit independently derived from the group consisting of aliphatic and alicylic compounds carrying at least two N═C═O groups, and (ii) at least one structural unit selected from the group consisting of uretdione, isocyanurate, biuret, urea, carbodiimide, uretonimine, urethane, allophanate, oxadiazinetrione and iminooxadiazinedione.

Even more preferably, component F is at least one oligomer or polymer carrying more than one N═C═O group and comprising (i) at least one unit independently derived from the group consisting of aliphatic and alicylic compounds carrying at least two N═C═O groups, and (ii) at least one isocyanurate structural unit.

Most preferably, component F is at least one oligomer or polymer carrying more than one N═C═O group and comprising (i) at least one unit independently derived from the group consisting of hexamethylene-1,6-diisocyanate, 4,4′- di(isocyanatocyclohexyl)methane, 2,4′-di(isocyanatocyclohexyl)methane and 1-isocyanato-3,3,5-trimethyl-5-(isocyanatomethyl)cyclohexane (isophorone diisocyanate), and (ii) at least one isocyanurate structural unit.

In particular, component F is at least one oligomer or polymer carrying more than one N═C═O group and comprising (i) at least one unit derived from hexamethylene-1,6-diisocyanate and (ii) at least one isocyanurate structural unit.

The molar ratio of N═C═O groups of polyisocyanate (F) to the OH groups of all components of the organic solvent-based two component coating composition, including polyester polyols of the present invention and components D, is from 50-150%, preferably 80 to 120%. A ratio of 100% is also referred to as so-called “index 100”.

The organic solvent-based two-component coating composition comprises at least one organic solvent.

Suitable organic solvents are esters, ketones, amides, ethers and aromatic hydrocarbons and mixtures thereof.

Examples of esters of are ethyl acetate, butyl acetate, 1-methoxy-2-propyl acetate, 2-butoxy ethyl acetate (butyl gycol acetate), propylene glycol diacetate, ethyl 3-ethoxypropionate, 3-methoxybutyl acetate, butyldiglycol acetate and propylene carbonate. Examples of ketones are acetone, methyl ethyl ketone and methyl isobutyl ketone. Examples of amides are dimethylformamide (DMF) and N-methyl pyrrolidone (NMP). Example of ethers are glycol ethers such as dipropylene glycol dimethylether, and cyclic ethers such as tetrahydrofuran and 1,4-dioxane. Examples of aromatic hydrocarbons are xylene and Solvesso 100.

A preferred organic solvent is an ester are or mixtures thereof. A more preferred organic solvent is an ester of a C₁₋₆-alkanoic acids with a C₁₋₆-alkanol such as butyl acetate and ethyl acetate. A particularly preferred organic solvent is butyl acetate.

The organic solvent-based two-component coating composition, preferably, also comprises at least one catalyst.

Examples of catalysts are organic bases, organic acids, organic metal compounds and inorganic metal salts.

Examples organic bases are amines such as diazobicyclo[2.2.2]octane (DABCO), amidine or guanidine-type compounds such as 1,5,7-triazabicyclo[4.4.0]dec-5-ene (TBD), N-methyl-1,5,7-triazabicyclododecene (MTBD), 1,8-diazabicyclo[5.4.0]undec-7-ene (DBU) and N-heterocyclic carbenes such as1,3-bis(ditert-butyl)imidazole-2-ylidene.

Examples of organic acids are organic sulfonic acids such as methylsulfonic acid and trifluoromethylsulfonic acid, and phosphonic acids such as diphenylphosphonic acid.

Examples of organic metal compounds are organic antimony compounds, organic bismuth compound, organic germanium compounds, organic tin compounds, organic lead compounds, organic aluminium compounds, organic zinc compounds, organic mercury compounds, organic copper compounds, organic nickel compounds, organic cobalt compounds, organic manganese compounds, organic molybdenum compounds, organic vanadium compounds, organic titanium compounds, organic zirconium compounds and organic cesium compounds.

Examples of organo tin compounds are organo tin(II) compounds such as tin(II) diacetate, tin(II) dioctoate, tin(II) bis(2-ethylhexanoate) and tin(II) dilaurate, as well as dialkyltin(IV) compounds such as dimethyltin(IV) diacetate, dibutyltin(IV) diacetate, dibutyltin(IV) dibutyrate, dibutyltin(IV) bis(2-ethylhexanoate), dibutyltin(IV) dilaurate, dibutyltin(IV) maleate, dioctyltin(IV) dilaurate and dioctyltin(IV) diacetate.

Examples of an organo zinc compounds are zinc(II) dioctoate and zinc(II) acetylacetonate. An example of an organo bismuth compound is bismuth(III) tris(neodecanoate).

Examples of organo zirconium compounds are zirconium(IV) tetrakis(acetylacetonate), zirconium (IV) tetrakis(2,4-pentandionate) and zirconium(IV) tetrakis(2,2,6,6-tetramethyl-3,5-heptanedionate).

An example of an organo iron compound is iron(III) tris(acetylacetonate). An example of an organo titanium compound is titanium(IV) tetrakis(acetylacetonate). An example of an organo manganese compound is manganese(III) tris(acetylacetonate). An example of an organo nickel compound is nickel(11) bis(acetylacetonate). Examples of an organo cobalt compounds are cobalt(11) bis(acetylacetonate) and cobalt (III) tris(acetylacetonate). Examples of organic molybdenum compounds are molybdenum(II) bis(acetylacetonate) and molybdenum dioxide tetramethylheptadionate. Examples of an organic cesium compound is cesium propionate and cesium 2-ethylhexanoate.

Examples of inorganic metal salts are lithium molybdate, lithium tungstate and cesium phosphate.

Preferably the catalyst is an organic metal compound. More preferably, the catalyst is an organic metal compound selected from the group consisting of organic tin compounds, organic zinc compounds, organic zirconium compounds and organic bismuth compounds. Even more preferably, the catalyst is selected from the group consisting of dimethyltin(IV) diacetate, dibutyltin(IV) dibutyrate, dibutyltin(IV) bis(2-ethylhexanoate), dibutyltin(IV) dilaurate, dioctyltin(IV) dilaurate, zinc(II) dioctoate, zirconium(IV) tetrakis(acetylacetonate), zirconium(IV) tetrakis(2,2,6,6-tetramethyl-3,5-heptanedionate) and bismuth(III) tris(neodecanoate). Most preferably, the catalyst is dibutyltin(IV) dilaurate.

The catalyst is usually used in an amount in the range of 50 to 10000 ppm, preferably 50 to 5000 ppm, more preferably 100 to 1000 ppm, based on the weight of all OH-group carrying components of the composition of the present invention.

The organic solvent-based two component coating composition can comprise a pigment and/or a dye.

Pigments can be organic or inorganic absorption pigments or organic or inorganic effect pigments.

Examples of organic absorption pigments are azo pigments, phthalocyanine pigments, quinacridone pigments, and pyrrolopyrrole pigments. Examples of inorganic absorption pigments are iron oxide pigments, titanium dioxide and carbon black.

Effect pigments are all pigments which exhibit a platelet-shaped construction and give a surface coating specific decorative color effect. The effect pigments can be pure metallic effect pigments such as aluminium, iron or copper effect pigments, interference effect pigments such as titanium dioxide-coated mica effect pigments, iron oxide-coated mica effect pigments, mixed oxide-coated mica effect pigments and metal oxide-coated aluminium effect pigments, or liquid-crystal effect pigments.

Examples of dyes are azo, azine, anthraquinone, acridine, cyanine, oxazine, polymethine, thiazine and triarylmethane dyes.

The organic solvent-based two component coating composition can comprise further additives such as defoamers, leveling agents, dispersing agents, grinding agents, light stabilizers, antistatic agents, flame retardants, thickeners, thixotropic agents, surface-active agents, viscosity modifiers, plasticizers, chelating agents, and fillers.

The additives are known in the art.

An example of a defoamer is EFKA® PB 2744. An example of a levelling agent is EFKA® WE 3050. An example of a dispersing agent is EFKA® PX 4330. An example of a grinding agent is Laropal® A-8L, a condensation product of urea and aliphatic aldehyde.

Examples of light stabilizers are UV absorbers and hindered amine light stabilizers (HALS).

Examples of UV absorbers are benzotriazoles such as benzenepropanoic acid, 3-(2H-benzotriazol-2-yl)-5-(1,1-dimethylethyl)-4-hydroxy ester and α-[3-[3-(2H-benzotriazol-2-yl)-5-(1,1-dimethylethyl)-4-hydroxyphenyl]-1-oxopropyl]-ω-hydroxpoly(oxo-1,2-ethanediyl), as well as benzophenones such as 2-hydroxy-4-n-octoxy benzophenone.

Examples of hindered amine light stabilizers are 2,2,6,6-tetramethylpiperidine, 2,6-di-tert-butylpiperidine, bis(2,2,6,6-tetramethyl-4-piperidyl) sebacate, bis(1,2,2,6,6-pentamethyl-4-piperidinyl) [[3,5-bis(1,1-dimethylethyl)-4-hydroxyphenyl]methyl]butylmalonate, bis(1,2,2,6,6-pentamethyl-4-piperidinyl) sebacate, methyl(1,2,2,6,6-pentamethyl-4-piperidinyl) sebacate and decanedioic acid, bis(1-octyloxy- 2,2,6,6-tetramethyl-4-piperidinyl) ester.

Examples of thickeners are hydroxymethyl cellulose and bentonite.

An example of a chelating agent is ethylenediamine tetraacetic acid.

Examples of fillers are silica gel, kieselgur, talc, calcium carbonate, kaolin, barium sulfate, magnesium silicate, aluminium silicate, siliceous earth, crystalline silicon dioxide, amorphous silica, aluminium oxide, microspheres or hollow microspheres made, for example, of glass, ceramic or polymers, urea-formaldehyde condensates, micronized polyolefin wax and micronized amide wax. Preferred fillers are siliceous earth, talc, aluminium silicate, magnesium silicate and calcium carbonate.

The organic solvent-based two-component coating composition can be prepared by mixing the first component (K1) with the second component (K2) in the presence of at least one organic solvent. At least one catalyst or further additives can be present when mixing the first component (K1) with the second component (K2), or added after mixing the first component (K1) with the second component (K2).

The flow time of the solvent-based two-component coating composition can be adjusted by addition of at least one organic solvent. This organic solvent can be the organic solvent already used as organic solvent in the first component K1. The flow time can be, for example adjusted so that the flow time is in the range of 10 to 50 seconds, preferably in the range of 20 to 35 seconds according to DIN EN 53211,1987 using a flow cup having a 4 mm hole diameter.

The amount of polyester polyols of the present invention is preferably in the range of 0.5 to 20 weight %, more preferably in the range of 1 to 17 weight %, and most preferably in the range of 2 to 15 weight % based on the weight of the organic solvent-based two-component coating composition.

The amount of polymer carrying more than one OH group, which are different from the polyester polyol of the present invention, (D), if present, are preferably 1 to 40 weight %, more preferably between 5 and 35 weight %, and most preferably between 10 and 25 weight % based on the weight of the organic solvent-based two-component coating composition.

The weight ratio of the polyester polyols of the present invention to the polymers carrying more than one OH group, which are different from the polyester polyols of the present invention, (D) in the first component (K1) of the organic solvent-based two component coating composition is preferably in the range of 0.01/1 to 2/1, preferably in the range of 0.1/1 to 1/1, more preferably, in the range of 0.15/1 to 0.65/1, most preferably in the range of 0.25/1 to 0.5/1.

The amount of compounds, oligomers or polymers carrying more than one N═C═O group or blocked N═C═O group (F) is preferably in the range of 1 to 40 weight %, more preferably in the range of 5 and 30 weight %, and most preferably in the range of 5 to 20 weight % based on the weight of organic solvent-based two-component coating composition.

The equivalent ratio of NCO groups of the compounds, oligomers or polymers carrying more than one N═C═O group or blocked N═C═O group (F) to the OH groups of the polyester polyols of the present invention and, if present, of the polymer carrying more than one OH group, which are different from the polyester polyol of the present invention, (D) is preferably in the range of to 1.2/0.8, more preferably in the range of 0.9/1.1 to 1.1/0.9.

In one preferred embodiment the organic solvent-based two-component coating composition is a pigmented organic solvent-based two-component coating composition comprising
a) a first component (K1) comprising (i) the polyester polyol of the present invention, and (ii) at least one polymer carrying more than one OH group, which is different from the polyester polyol of the present invention, (D),
b) a second component (K2) comprising (i) at least one compound, oligomer or polymer carrying more than one N═C═O group or blocked N═C═O group (F), wherein the first component (K1) and/or the second component (K2) also comprises at least one organic solvent, at least one catalyst, at least one pigment, and at least one additive.

The sum of polyester polyols of the present invention, polymers carrying more than one OH group, which are different from the polyester polyol of the present invention, (D) and of compounds, oligomers or polymers carrying more than one N═C═O group or blocked N═C═O group (F) preferably represents from 5 to 80 weight %, more preferably from 10 to 60 weight %, and most preferably from 20 to 45 weight % of the pigmented organic solvent-based two-component coating composition.

The pigment preferably represents from 5 to 80 weight %, more preferably from 20 to 60 weight %, and most preferably from 30 to 40 weight % of the pigmented organic solvent-based two-component coating composition.

The organic solvent preferably represents from of 1 to 60 weight %, more preferably in the range of 5 to 45 weight %, and most preferably in the range of 15 to 35 weight % of the pigmented organic solvent-based two-component coating composition.

The additive preferably represents from 0.5 to 40 weight %, more preferably from 2 to 20 weight % and most preferably from 3 to 15 weight % of the pigmented organic solvent-based two-component coating composition.

The polyester polyol, the one polymer carrying more than one OH group, which is different from the polyester polyol of the present invention, (D), at least one compound, oligomer or polymer carrying more than one N═C═O group or blocked N═C═O group (F), the organic solvent and the catalyst, the pigment and the additive are as described above.

The pigment is preferably an inorganic absorption pigments, and more preferably titanium dioxide.

The additive is preferably selected from the group consisting of defoamer agent, levelling agent, dispersing agent and grinding agent.

The pigmented organic solvent-based two-component coating composition can be prepared by preparing the first component (K1) by mixing (i) the polyester polyol of the present invention, and (ii) the at least one polymer carrying more than one OH group, which is different from the polyester polyol of the present invention, (D), adding organic solvent, catalyst, pigment paste and additives such as defoaming agents and levelling agents to the first component (K1), and then adding the second component (K2).

The pigment paste usually comprises the pigment, organic solvent and additives such as dispersing agents and grinding agents and can be prepared by stirring the ingredients at high speed in the presence of glass beads, followed by removal of the glass beads. If the dispersing or grinding agents are also polymers carrying more than one OH group, which polymers are different from the polyester polyol of the present invention, the dispersing or grinding agents are considered to represent a polymer carrying more than one OH group, which is different from the polyester polyol of the present invention, (D).

The flow time of the pigmented organic solvent-based two-component coating composition can be adjusted by addition of at least one organic solvent. This organic solvent can be the organic solvent already used as organic solvent in the first component K1. The flow time can be, for example adjusted so that the flow time is in the range of 10 to 50 seconds, preferably in the range of 15 to 35 seconds according to DIN EN 53211,1987 using a flow cup having a 4 mm hole diameter

Also part of the present invention is a substrate coated with the organic solvent-based two component composition of the present invention.

Examples of substrates are wood, wood veneer, paper, cardboard, paperboard, textile, film, leather, nonwoven, plastics surfaces, glass, ceramic, mineral building materials, such as molded cement blocks and fiber-cement slabs, and metals, which in each case are optionally precoated or pretreated. A preferred substrate is metal, which is optionally precoated or pretreated.

Also part of the present invention is a process for coating a substrate with the organic solvent-based two component composition of the present invention which comprises the step of applying the organic solvent-based two component composition to the substrate.

The organic solvent-based two-component coating composition of the present invention can be applied to the substrate by methods common in the art such as by draw down bar, spraying, troweling, knifecoating, brushing, rolling, rollercoating, flowcoating and laminating.

Following the application of the organic solvent-based two-component coating composition of the invention, the composition of the present invention is cured at a temperature in the range of 15 to 140° C., preferably in the range of 20 to 100° C.

The thickness of the “wet” layer formed from the organic solvent-based two-component coating composition of the present invention is usually in the range of 20 to 5000 μm, preferably in the range of 50 to 500 μm, more preferably in the range of 100 to 250 μm. After curing, the thickness of the layer is usually in the range of 10 to 500 μm, preferably in the range of 15 to 200 μm, more preferably in the range of 20 to 100 μm.

Substrates coated with the organic solvent-based two-component coating composition of the present invention can, for example, be part of automotives, large vehicles, aircrafts, utility vehicles in agriculture and construction, bridges, buildings, power masts, tanks, containers, pipelines, power stations, chemical plants, ships, cranes, posts, sheet piling, valves, pipes, fittings, flanges, couplings, halls, roofs, furniture, windows, doors, woodblock flooring, cans, coils and floors.

The organic solvent-based two-component coating composition of the present invention can, for example, be used as pigmented organic solvent-based two-component coating composition.

Also part of the present invention is a coating layer on a substrate formed from the the organic solvent-based two component composition of the present invention.

Also part of the present invention is the use of the composition of the present invention in coating composition suitable for preparing coatings of automotives, large vehicles and of utility vehicles in agriculture and construction.

The organic solvent-based two-component compositions of the present invention are advantageous in that the polyester polyol of the first component (K1) of the composition is at least partly derived from biomonomers.

The organic-solvent based two-component coating compositions of the present invention are also advantageous in that the compositions show a good drying behavior, in particular a short cotton wool drying time. At the same time, the coatings formed from the organic solvent-based two-component coating composition of the present invention show acceptable solid contents and mechanical properties such as a high pendulum hardness.

The organic-solvent based two-component coating compositions of the present invention are also advantageous in that the coatings formed from the organic-solvent based two-component coating compositions also show a high chemical resistance.

The organic-solvent based two-component coating compositions of the present invention are also advantageous in that the coatings formed from the organic-solvent based two-component coating compositions also show a high water resistance.

The organic-solvent based two-component coating compositions comprising additionally polymers carrying more than one OH group, which are different from the polyester polyol of the present invention, (D) are in particular advantageous in that the coating layer formed from the compositions is not sticky. Thus, the organic-solvent based two-component coating compositions comprising additionally polymers carrying more than one OH group, which are different from the polyester polyol of the present invention, (D) are not suitable as adhesive compositions.

### EXAMPLES

Description of Test Methods

The weight average molecular weight Mw and number average molecular weight Mn were determined using gel permeation chromatography calibrated to a polystyrene standard.

The glass transition temperature (Tg) was determined using differential scanning calorimetry.

The hydroxyl number was determined according to DIN53240, 2016.

The acid number was determined according to DIN53402, 1990.

The solid content of solutions comprising polyester polyol were measured using a moisture analyzer (Mettler Toledo HB43-S Moisture Analyzer) at 160° C. until constant mass was reached.

The solid content of white pigmented coating compositions comprising the polyester polyol solutions were calculated based on the measured solid content of the polyester polyol solutions.

The viscosity was determined using a cone plate viscosimeter set to a shear rate of 100 s⁻¹ at 23° C.

Cotton wool drying time: The coating composition was applied with a draw down bar on a glass plat yielding a wet film thickness of 150 μm. After film application, a frayed cotton wool was swept without pressure across the surface of the coating every 5 to 10 minutes. At the beginning, cotton fibers were sticking to the coating. The time when no fibers remained attached to the coating, is referred to as the cotton wool drying time.

Pendulum hardness [osc.]: The coating composition was applied with a draw down bar having a gap of 150 μm on a 4 mm thick glass plate, which has been cleaned with acetone before, yielding a wet film. The pendulum hardness was measured according to DIN EN ISO 1522:2006 using the Konig pendulum.

### Comparative Example 1

Preparation of a Solution Comprising a Polyester Polyol Comp1 Prepared from HHPA and TMP

Cyclohexane-1,2-dicarboxylic acid anhydride (mixture of isomers) (HHPA) and 1,1,1-trime-thylolpropane (TMP) were mixed in a molar ratio as indicated in table 1 and slowly heated to 160° C. under a steady stream of nitrogen. When the reaction mixture reached 135° C., an exothermic reaction was observed. The reaction mixture was kept at 160° C. for 30 min, and then heated to 180° C. Water was removed by distillation. The reaction was monitored by the titration of the acid number and cooled down to 80° C. when the desired value was reached. Butyl acetate was added to the melt to yield a solution comprising a polyester polyol comp 1 with a solid content as indicated in table 1. The solid content, the hydroxyl number, the acid number, the glass temperature (Tg) the number average molecular weight (Mn), the weight average molecular weight (Mw) of the polyster polyol comp1 and the viscosity of the solution of the polyster polyol comp 1 were determined according to the methods described in the section above titled “Description of test methods” and are also shown in table 1.

### Comparative Example 2

Preparation of a Solution Comprising Polyester Polyol Comp2 Prepared from HHPA, CHDM and THEIC

Cyclohexane-1,2-dicarboxylic acid anhydride (mixture of isomers) (HHPA), 1,4-bis(hydroxymethyl)cyclohexane (CHDM) and 1,3,5-tris(2-hydroxyethyl) isocyanurate (THEIC) were mixed in a molar ratio as indicated in table 1 and slowly heated to 160° C. under a steady stream of nitrogen. When the reaction mixture reached 135° C., an exothermic reaction was observed. The reaction mixture was kept at 160° C. for 30 min, and then heated to 180° C. Water was removed by distillation. The reaction was monitored by the titration of the acid number and cooled down to 80° C. when the desired value was reached. Butyl acetate was added to the melt to yield a solution comprising the polyester polyol comp2 with a solid content as indicated in table 1. The solid content, the hydroxyl number, the acid number, the glass temperature (Tg) the number average molecular weight (Mn) and the weight average molecular weight (Mw) of polyster polyol of polyster polyol comp2 and the viscosity of the solution of polyster polyol comp2 were determined according to the methods described in the section above titled “Description of test methods” and are also shown in table 1.

### Example 1

Preparation of Solutions Comprising Hyperbranched Polyester Polyols 1a, 1b and 1c Comprising Isosorbide

Cyclohexane-1,2-dicarboxylic acid anhydride (mixture of isomers) (HHPA), isosorbide and 1,3,5-tris(2-hydroxyethyl) isocyanurate (THEIC) were mixed in a molar ratio as indicated in table 1. 500 ppm of titanium(IV) n-butoxide, based on the weight of the reaction mixture, was added. The reaction mixture was slowly heated to 160° C. under a steady stream of nitrogen. When the reaction mixture reached 135° C., a light exothermic reaction was observed. The reaction mixture was kept at 160° C. for 30 min, and then heated to 180° C. Water was removed by distillation. The reaction was monitored by the titration of the acid number and cooled down to 80° C. when the desired value was reached (51 mg KOH/g for 1a, 121 mg KOH/g for 1b and 53 mg KOH/g for 1c). Butyl acetate was added to the melt to yield a solution comprising the polyester polyol 1a, 1b and 1c, respectively, with a solid content as indicated in table 1. The solid content, the hydroxyl number, the acid number, the glass temperature (Tg) the number average molecular weight (Mn) and the weight average molecular weight (Mw) of polyster polyol 1a, 1b and 1c and the viscosity of the solution of polyster polyol 1a, 1b and 1c, respectively, were determined according to the methods described in the section above titled “Description of test methods” and are also shown in table 1.

TABLE 1

| | polyester polyol | | | | |
|---|---|---|---|---|---|
| monomers | comp1 | Comp2 | 1a | 1b | 1c |
| HHPA | 50 | 47.1 | 47.1 | 53.3 | 53.3 |
| CHDM | — | 17.7 | — | — | — |
| isosorbide | — | — | 17.7 | 20.0 | 20.0 |
| THEIC | — | 35.3 | 35.3 | 26.7 | 26.7 |
| TMP | 50 | — | — | — | — |
| properties | | | | | |
| Hydroxyl number | 285 | 178 | 189 | 149 | 77 |
| [mg KOH/g] | | | | | |
| acid number [mg | 85 | 26 | 51 | 121 | 53 |
| KOH/g] | | | | | |
| Mn [g/mol] | 1050 | 750 | 530 | 450 | 970 |
| Mw [g/mol] | 1650 | 1580 | 1030 | 680 | 3080 |
| Tg [° C.] | 9 | 47.3 | 41.2 | 36.6 | 49.1 |
| solid content [%] | 69.1 | 67.4 | 64.4 | 64.1 | 67.8 |
| Viscosity | 3851 | 5100 | 4650 | 1740 | 7900 |
| [mPa × s] | | | | | |

### Example 2

Preparation of a White Pigment Paste

20 g Laropal® A-8L (a grinding resin, 80% solution of a condensation product of urea and aliphatic aldehyde in 1-methoxy-2-propyl acetate, hydroxyl number: 90 mg KOH/g, available from BASF), 3.0 g 1-methoxy-2-propyl acetate and 3.0 g EFKA® PX 4330, a high molecular weight dispersing agent available from BASF, were mixed using a lab stirrer. The speed of the stirrer was slowly increased to 4000 rpm and kept at that speed for 5 minutes. 74.0 g Kronos® 2310, a white pigment with a white pigment index 6, was slowly added to the mixture under stirring. The speed of the stirrer was slowly increased to 5000 rpm and kept at that speed for 10 minutes. Then, the mixture was placed in a grinding mill. 150 g glass beads with a diameter in the range from 0.75 to 1 mm were added and the mixture was grinded at a speed of 5500 rpm for 30 minutes. Afterwards, the white pigment paste was separated from the glass beads using compressed air.

### Example 3

Preparation of a white pigmented coating composition comprising the polyester polyols comp1, comp2, 1a, 1b and 1c, respectively, and application of the coating compositions on a glass plate

0.647 g of a 1 wt % solution of dibutyltin(IV) dilaurate (catalyst) in butyl acetate was added in a 100 mL glass jar. Then, 0.388 g EFKA® PB 2744 (a defoamer available from BASF) was added, followed by 0.516 g EFKA® WE 3050 (a levelling agent available from BASF).

Then, 18 g of of Setalux® 1907 BA-75 (a 75 weight % solution of an acrylic polyol with 4.5% OH groups calculated on non-volatiles and available from Allnex in butyl acetate) was combined with the amount of the solution of polyester polyol of comparative example 1, comparative example 2, example 1 and example 2, respectively, comprising the polyester polyol compl, comp2, 1a, 1b and 1c, respectively, containing 4.5 g solids, and the obtained mixture comprising the acrylic polyol and polyester polyol was added to the mixture above comprising catalyst, defoamer and levelling agent.

49.10 g of the white pigment paste of example 3 was added to the mixture. The mixture was stored for 16 h. Then, 10 g butyl acetate was added, and the mixture was stirred using a lab stirrer with a 35 mm disc at a speed of 750 rpm for 5 minutes.

Basonat® HI 2000 NG (solvent-free, aliphatic polyisocyanate) at an index of 100 (with respect to the OH groups of Setalux® 1907 BA-75, polyester polyol, and Laropal® A-8L) was added to the mixture. The mixture was stirred using a lab stirrer with a 35 mm disc at a speed of 750 rpm for 10 minutes. Subsequently, butyl acetate was added in an amount that the viscosity measured with a cone plate viscosimeter corresponds to 200 mPa×s. After waiting for 10 min, the pigmented coating composition was ready to use.

After cleaning glass substrates properly with acetone, the pigmented coating compositions were applied with a draw down bar with a wet film thickness of 200 μm. The dry film thickness was approximately 60 μm.

The solid content, the cotton wool drying time and the pendulum hardness [osc.] of the white pigmented coating compositions comprising polyester polyols comp1, comp2, 1a, 1b and 1c, respectively, were determined as described above in the section titled “Description of Test Methods” and are shown in table 2.

TABLE 2
¹Pendulum Hardness.

| | Pigmented coating composition | | | | |
|---|---|---|---|---|---|
| | comprising polyester polyol | | | | |
| monomers | comp1 | Comp2 | 1a | 1b | 1c |
| HHPA | 50 | 47.1 | 47.1 | 53.3 | 53.3 |
| CHDM | — | 17.7 | — | — | — |
| isosorbide | — | — | 17.7 | 20.0 | 20.0 |
| THEIC | — | 35.3 | 35.3 | 26.7 | 26.7 |
| TMP | 50 | — | — | — | — |
| properties | | | | | |
| solid content [%] | 76.4 | 76.9 | 75.7 | 75.3 | 75.7 |
| cotton wool drying | 160 | 90 | 60 | 60 | 50 |
| time [min] | | | | | |
| PH¹ [osc.] after 1 d at | 14 | 14 | 13 | 15 | 14 |
| RT | | | | | |
| PH¹ [osc.] after 2 d at | 33 | 29 | 26 | 25 | 28 |
| RT | | | | | |
| PH¹ [osc.] after 7 d at | 38 | 38 | 36 | 30 | 35 |
| RT | | | | | |
| PH¹ [osc.] after 7 d at | 52 | 53 | 50 | 45 | 48 |
| RT + 15 h at 60° C.) | | | | | |
| PH¹ [osc.] after 15 | 33 | 35 | 38 | 41 | 37 |
| min at RT + 30 min at | | | | | |
| 60° C. + 1 d at RT | | | | | |
| PH¹ [osc.] after 15 | 60 | 59 | 81 | 65 | 59 |
| min at RT + 30 min at | | | | | |
| 60° C. + 6 d at RT | | | | | |
| PH¹ [osc.] after 15 | 88 | 82 | 95 | 88 | 80 |
| min at RT + 30 min at | | | | | |
| 80° C. + 6 d at RT | | | | | |

Table 2 shows that inventive white pigmented organic solvent-based two-component coating compositions comprising polyester polyol 1a, 1b and 1c (comprising isosorbide), respectively, show a shorter cotton wool drying time than comparative white pigmented organic solvent-based two-component coating compositions comprising polyester polyol compl (comprising trimethylolpropane instead of isosorbide) or polyester polyol comp2 (comprising 1,4-bis(hydroxymethyl)cyclohexane (CHDM) instead of isosorbide). At the same time the inventive white pigmented organic solvent-based two-component coating compositions comprising polyester polyol 1a, 1b and 1c, respectively, have a comparable solid content and comparable pendulum hardness than the coating composition comprising polyester polyol comp2 or polyester polyol comp1.

1-17. (canceled)

18. A polyester polyol comprising units derived from
a) at least one component (A) carrying at least one COOH group or a derivative thereof, wherein component (A) comprises
(i) at least one compound carrying two COOH groups or derivatives thereof (A1), and
b) at least one component (B) carrying at least one OH group and no COOH group, wherein component (B) comprises
(ii) at least one compound or oligomer carrying at least three OH groups and no COOH group (B1),
(iii) at least one compound carrying two OH groups (B2) selected from the group consisting of 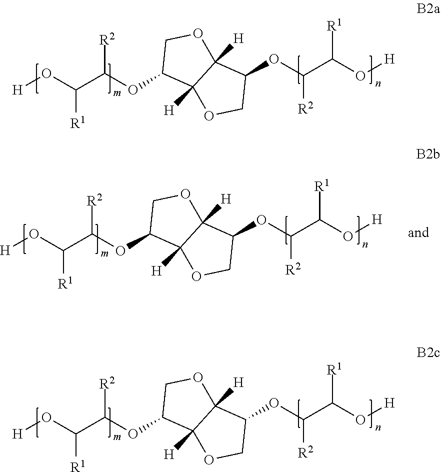
wherein n and m are independently from each other 0 or 1, and R¹ and R² are independently selected from the group consisting of H, CH₃ and CH₂CH₃, and
(iv) optionally at least one compound, oligomer or polymer carrying two OH groups and no COOH group, which is different from B2 (B3),
wherein the molar ratio of the OH groups of components B1 to the sum of OH groups of components B1, B2 and B3 is in the range of 25 to 90%.

19. The polyester polyol of claim 18, wherein compound A1 is at least one aliphatic or alicyclic compound carrying two COOH groups or a derivative thereof

20. The polyester polyol of claim 18, wherein compound or oligomer B1 carries three OH groups and no COOH group.

21. The polyester polyol of claim 18, wherein the compound carrying two OH groups (B2) is a compound of formula 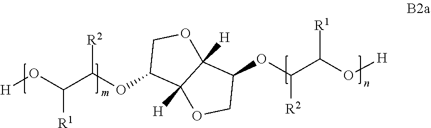
wherein n and m are independently from each other 0 or 1, and R¹ and R² are independently selected from the group consisting of H, CH₃ and CH₂CH_{3,} with the proviso that at least one of R¹ or R² is H.

22. The polyester polyol of claim 18, wherein the molar ratio of the OH groups of components B1 to the sum of OH groups of components B1, B2 and B3 is in the range of 50% to 90%.

23. The polyester polyol of claim 22, wherein the molar ratio of the OH groups of components B1 to the sum of OH groups of components B1, B2 and B3 is in the range of from to 85%.

24. The polyester polyol of claim 18, wherein the molar ratio of the sum of OH groups of all components A and B to the sum of COOH groups of all components A is in the range of 1.05/1 to 3/1.

25. The polyester polyol of claim 18, wherein the polyester polyols have a hydroxyl number in the range of 50 to 400 mg KOH/g.

26. The polyester polyol of claim 18, wherein the polyester polyols have an acid number in the range of 1 to 200 mg KOH/g.

27. The polyester polyol of claim 18, wherein the polyester polyols have a number average molecular weight Mn in the range of 400 to 5000 g/mol.

28. The polyester polyols of claim 18, wherein the polyester polyols have a weight average molecular weight Mw in the range of 400 to 15000 g/mol.

29. A solution comprising the polyester polyols of claim 18, and at least one organic solvent.

30. An organic solvent-based two-component coating composition comprising
a) a first component (K1) comprising (i) at least one polyester polyol of claim 18, and (ii) optionally at least one polymer carrying more than one OH group, which is different from the polyester polyol of the present invention, (D)
and
b) a second component (K2) comprising (i) at least one compound, oligomer or polymer carrying more than one N═C═O group or blocked N═C═O group (F).

31. The organic solvent-based two-component coating composition of claim 30, wherein the polymer carrying more than one OH group, which is different from the polyester polyol of the present invention, (D) is present and is at least one (meth)acrylic polymer carrying more than one OH group.

32. The organic solvent-based two-component coating composition of claim 30, wherein the first component (K1) and/or the second component (K2) also comprises at least one organic solvent, at least one catalyst, at least one pigment, and at least one additive.

33. A substrate coated with the organic solvent-based two component composition of claim 30.

34. A coating layer on a substrate formed from the organic solvent-based two component composition of claim 30.
